(19) United States
(12) Patent Application Publication
(10) Pub. No.: US 2025/0391018 A1
(43) Pub. Date: Dec. 25, 2025

(54) COMPUTE SYSTEM WITH SKIN LESION MEASUREMENT MECHANISM AND METHOD OF OPERATION THEREOF

(71) Applicant: BelleTorus Corporation, Cambridge, MA (US)

(72) Inventors: Thi Thu Hang Nguyen, Toulouse (FR); Susely Figueroa Iglesias, Frouzins (FR); Paul Fricker, Toulouse (FR)

(21) Appl. No.: 19/058,468

(22) Filed: Feb. 20, 2025

Related U.S. Application Data
(60) Provisional application No. 63/663,090, filed on Jun. 22, 2024.

(51) Int. Cl. G06T 7/00; G06T 7/11; G06T 7/62; G16H 15/00

(52) U.S. Cl. CPC G06T 7/0012; G06T 7/11; G06T 7/62; G16H 15/00; G06T 2207/20084; G06T 2207/30088; G06T 2207/30096

(57) ABSTRACT

A method of operation of a compute system includes: receiving a patient image including a skin lesion and a fixed ruler, generating a correlated ruler by segmenting the fixed ruler including translating pixel-to-millimeter to the patient image, calculating a skin lesion area by segmenting the skin lesion, and generating a skin lesion output report, including an image of the skin lesion, the correlated ruler and the skin lesion area, for displaying on a device.

### CROSS REFERENCE TO RELATED APPLICATION(S)

This claims the benefit of U.S. Provisional Patent Application Ser. No. 63/663,090 filed Jun. 22, 2024, and the subject matter thereof is incorporated herein by reference thereto.

### TECHNICAL FIELD

An embodiment of the present invention relates generally to a compute system, and more particularly to a system with an artificial intelligence (AI) based skin lesion measurement mechanism.

### BACKGROUND

In dermatology, it is particularly useful to evaluate the risk of skin lesions by using dermoscopes (also known as dermatoscopes) due to several compelling reasons rooted in the enhanced diagnostic capabilities of these instruments. Along with the magnification and polarization of the lesions, the ability to accurately measure skin lesions is a crucial aspect of dermoscopic examination, providing significant benefits for assessing and managing of dermatological conditions.

Thus, a need still remains for a compute system with a skin lesion measurement mechanism to provide an artificial intelligence (AI) based approach to measure skin pigmentation or lesions for monitoring, diagnosing, and prescribing skin ailment treatments. In view of the ever-increasing commercial competitive pressures, along with growing healthcare needs, healthcare expectations, and the diminishing opportunities for meaningful product differentiation in the marketplace, it is increasingly critical that answers be found to these problems. Additionally, the need to reduce costs, improve efficiencies and performance, and meet competitive pressures adds an even greater urgency to the critical necessity for finding answers to these problems.

Solutions to these problems have been long sought but prior developments have not taught or suggested any solutions and, thus, solutions to these problems have long eluded those skilled in the art.

### DISCLOSURE OF THE INVENTION

An embodiment of the present invention provides a method of operation of a compute system including: receiving a patient image including a skin lesion and a fixed ruler; generating a correlated ruler by segmenting the fixed ruler including applying a pixel-to-millimeter algorithm to the patient image; calculating a skin lesion area by segmenting the skin lesion; and generating a skin lesion output report, including an image of the skin lesion, the correlated ruler and the skin lesion area, for displaying on a device.

An embodiment of the present invention provides a compute system, including a control circuit, including a processor, configured to: receive a patient image, through a digital camera, including a skin lesion and a fixed ruler; generate a correlated ruler by segmenting the fixed ruler including applying a pixel-to-millimeter algorithm to pixels of the patient image; calculate a skin lesion area by segmenting the skin lesion; and generate a skin lesion output report, including an image of the skin lesion, the correlated ruler and the skin lesion area, for displaying on a device.

An embodiment of the present invention provides a non-transitory computer readable medium including instructions executable by a control circuit for a compute system performing functions including: receiving a patient image including a skin lesion and a fixed ruler; generating a correlated ruler by segmenting the fixed ruler including applying a pixel-to-millimeter algorithm to the patient image; calculating a skin lesion area by segmenting the skin lesion; and generating a skin lesion output report, including an image of the skin lesion, the correlated ruler and the skin lesion area, for displaying on a device.

Certain embodiments of the invention have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an example of a system architectural diagram of a compute system with a skin lesion measurement mechanism in an embodiment of the present invention

FIG. 2 is an example of a skin lesion area flow including the ruler segmentation module, the skin lesion segmentation module, and the skin lesion area rectangle module.

FIG. 3 depicts an example segmentation of the ruler segmentation module with the patient image input and the correlated ruler as the output.

FIG. 4 depict examples of fixed ruler types applied to dermoscopic images.

FIG. 5 depicts an example image for an annotation process that allows training the model of ruler segmentation by the ruler segmentation module.

FIG. 6 depicts an example comparison graph of skin lesion size estimation of four different annotators using fixed rulers in a dermoscope.

FIG. 7 is an exemplary block diagram of the compute system in an embodiment.

FIG. 8 is a flow chart of a method of operation of a compute system in an embodiment of the present invention.

### BEST MODE FOR CARRYING OUT THE INVENTION

In dermatology, measuring skin lesions is critical for monitoring changes over time. This longitudinal monitoring is vital for detecting changes in size, shape, or other characteristics that might indicate malignancy. For example, a skin lesion that grows rapidly over a short period could be a sign of melanoma or other skin cancer, necessitating prompt medical intervention. Consistent and precise measurements are required to ensure that even subtle changes are noted and acted upon.

The skin lesion's diameter is one of the four important features of the skin lesion. As an example, when the skin lesion is larger than 6 millimeters, a warning sign rises. Most modern dermoscopes have integrated a fixed scale that helps physicians estimate the size. The scale is only capable of detecting significant growth of the skin lesion, because the scale is fixed to the lens of the dermoscope causing a parallax error in the readings. Accurate size measurements, including the actual measurements of the skin lesion, are critical for diagnosing and tracking the growth of skin lesions, such as moles or melanoma. Embodiments can measure the skin lesion's dimensions and area by processing the integrated scale from the dermoscope. Embodiments include skin lesion segmentation model, ruler segmentation model, and the area rectangle model including a pixel-to-millimeter conversion algorithm. Embodiments have been evaluated with a test set of images with various correlated ruler types. The correlated ruler is the result of converting a fixed and unrelated ruler etched on the lens of the dermoscope to an actual measure of the viewed lesion.

The following embodiments are described in sufficient detail to enable those skilled in the art to make and use the invention. It is to be understood that other embodiments would be evident based on the present disclosure, and that system, process, or mechanical changes may be made without departing from the scope of an embodiment of the present invention.

In the following description, numerous specific details are given to provide a thorough understanding of the invention. However, it will be apparent that the invention may be practiced without these specific details. In order to avoid obscuring an embodiment of the present invention, some well-known circuits, system configurations, and process steps are not disclosed in detail.

The drawings showing embodiments of the system are semi-diagrammatic, and not to scale and, particularly, some of the dimensions are for the clarity of presentation and are shown exaggerated in the drawing figures. Similarly, although the views in the drawings for ease of description generally show similar orientations, this depiction in the figures is arbitrary for the most part. Generally, the invention can be operated in any orientation. The embodiments of various components as a matter of descriptive convenience and are not intended to have any other significance or provide limitations for an embodiment of the present invention.

The term “module” or “unit” or “circuit” referred to herein can include or be implemented as or include software running on specialized hardware, hardware, or a combination thereof in the present invention in accordance with the context in which the term is used. For example, the software can be machine code, firmware, embedded code, and application software. The software can also include a function, a call to a function, a code block, or a combination thereof. The term “model” can include software running on specific hardware structures to execute the analysis provided by the model.

Also, for example, the hardware can be gates, circuitry, processor, computer, integrated circuit, integrated circuit cores, memory devices, a pressure sensor, an inertial sensor, a microelectromechanical system (MEMS), passive devices, physical non-transitory memory medium including instructions for performing the software function, a portion therein, or a combination thereof to control one or more of the hardware units or circuits. Further, if a “module” or “unit” or a “circuit” is written in the claims section below, the “unit” or the “circuit” is deemed to include hardware circuitry for the purposes and the scope of the claims.

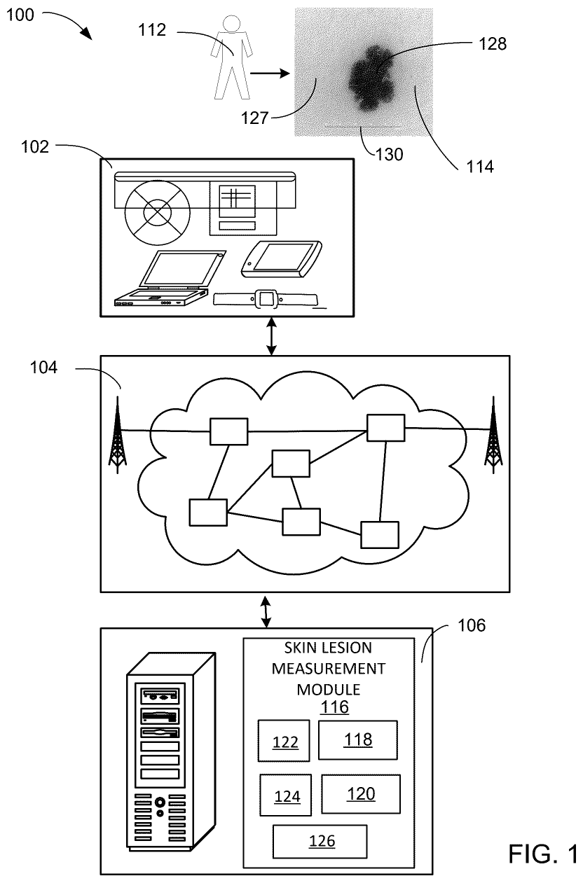

Referring now to FIG. 1, therein is shown an example of a system architecture diagram of a compute system 100 with a skin lesion measurement mechanism in an embodiment of the present invention. Embodiments of the compute system 100 provide standardized and accurate measurements to provide for a reproducible precise skin lesion measurement by a dermoscope.

The compute system 100 can include a first device 102, such as a dermoscope, a client, or a server, connected to a second device 106, such as a client or server. The first device 102 can communicate with the second device 106 through a network 104, such as a wireless or wired network.

For example, the first device 102 can be of any of a variety of skin lesion measuring devices, such as a dermoscope, a smart phone, a tablet, a cellular phone, personal digital assistant, a notebook computer, a wearable device, internet of things (IoT) device, or other multi-functional device capable of providing dermoscopic images. Also, for example, the first device 102 can be included in a device or a sub-system.

The first device 102 can couple, either directly or indirectly, to the network 104 to communicate with the second device 106 or can be a stand-alone device. The first device 102 can further be separate from or incorporated with a smart phone, a tablet computer, a laptop computer, a scanner, or other personal electronic devices.

For illustrative purposes, the compute system 100 is described with the first device 102 as a mobile device, although it is understood that the first device 102 can be different types of devices. For example, the first device 102 can also be a non-mobile computing device, such as a dermoscope, a server, a server farm, cloud computing, or a desktop computer.

The second device 106 can be any of a variety of centralized or decentralized computing devices. For example, the second device 106 can be a computer, grid computing resources, a virtualized computer resource, cloud computing resource, routers, switches, peer-to-peer distributed computing devices, or a combination thereof.

The second device 106 can be centralized in a single room, distributed across different rooms, distributed across different geographical locations, embedded within a telecommunications network. The second device 106 can couple with the network 104 to communicate with the first device 102. The second device 106 can also be a client type device as described for the first device 102.

For illustrative purposes, the compute system 100 is described with the second device 106 as a non-mobile computing device, although it is understood that the second device 106 can be different types of computing devices. For example, the second device 106 can also be a mobile computing device, such as notebook computer, another client device, a wearable device, or a different type of client device.

Also, for illustrative purposes, the compute system 100 is described with the second device 106 as a computing device, although it is understood that the second device 106 can be different types of devices. Also, for illustrative purposes, the compute system 100 is shown with the second device 106 and the first device 102 as endpoints of the network 104, although it is understood that the compute system 100 can include a different partition between the first device 102, the second device 106, and the network 104. For example, the first device 102, the second device 106, or a combination thereof can also function as part of the network 104.

The network 104 can span and represent a variety of networks. For example, the network 104 can include wireless communication, wired communication, optical, ultrasonic, or the combination thereof. Satellite communication, cellular communication, Bluetooth, Infrared Data Association standard (IrDA), wireless fidelity (WiFi), and worldwide interoperability for microwave access (WiMAX) are examples of wireless communication that can be included in the communication path. Ethernet, digital subscriber line (DSL), fiber to the home (FTTH), and plain old telephone service (POTS) are examples of wired communication that can be included in the network 104. Further, the network 104 can traverse a number of network topologies and distances. For example, the network 104 can include direct connection, personal area network (PAN), local area network (LAN), metropolitan area network (MAN), wide area network (WAN), or a combination thereof.

For example, the compute system 100 can provide the functions for a patient 112 with the first device 102, the second device 106, distributed between these two devices, or a combination thereof. Also, as examples, the compute system 100 can provide a mobile application for the patients 112, the clinicians, or a combination thereof. Further as an example, the compute system 100 can provide the functions via a web-browser based applications or a software to be executed on the first device 102, the second device 106, distributed between these two devices, or a combination thereof.

In one embodiment as an example, patient images 114 are taken and uploaded by the patient 112 and reviewed by the clinician. In this embodiment, the patient 112 launches the skin lesion measurement mechanism via the mobile application and logs into the account of the patient 112. The patient 112 can be prompted to upload or take images as the patient images 114. The compute system 100 can guide the patient 112 on photo guidelines for the patient images 114 and accepts or rejects the patient images 114 for retake based on a pre-specified criteria, including distance, quality, blur, or a combination thereof. The compute system 100 can also provide guides for the patient 112 on capturing videos as opposed to still photos. The patient images 114 can be selected from the video.

Once the patient images 114, as required for analysis, are successfully uploaded, the compute system 100 can send or load the patient images 114 to a skin lesion measurement module 116 for analysis. The skin lesion measurement module 116 will be described later. For brevity and clarity and as an example, the skin lesion measurement module 116 is shown as being executed in the second device 106 although it is understood that portions can operate on the first device 102, such as the mobile app or the web-browser based application, can operate completely on the first device 102, or a combination thereof. As a further example, the skin lesion measurement module 116 can include the artificial intelligence (AI) to operate an image quality checker 118, a skin lesion segmentation module 120, a ruler segmentation module 122, a skin lesion area rectangle module 124, and a skin lesion output module 126 displaying a skin lesion 128. The skin lesion measurement module 116 can be implemented in software running on specialized hardware, full hardware, or a combination thereof. The skin lesion measurement module 116 can be based on a convolutional neural network in a U-Net configuration executing an Inception-ResNet model.

The image quality checker 118 can be implemented in software running on specialized hardware, full hardware, or a combination thereof. The image quality checker 118 analyzes pixels 127 and metadata in the patient images 114 to detect the pre-specified criteria, including focal distance, quality, blur, type pf device used to capture the image, number of the pixels 127 in the image, fixed ruler type on lens, or a combination thereof. The image quality checker 118 can identify acceptable versions of the patient images 114 with clear visibility of the skin lesion 128, uniform focus throughout, and without visual obstructions, including cosmetics, dirt, or other exogenous pigments.

The skin lesion segmentation module 120 can be implemented in software running on specialized hardware, full hardware, or a combination thereof. The skin lesion segmentation module 120 analyzes the pixels 127 in the patient images 114 to detect areas in the patient images 114 that include the skin lesion 128 on a body part of the patient 112. The skin lesion segmentation module 120 can identify all of the skin lesion 128 in the patient images 114.

The ruler segmentation module 122 can be implemented in software running on specialized hardware, full hardware, or a combination thereof. The ruler segmentation module 122 can segment a fixed ruler 130 in the patient images 114 in order to generate a correlated ruler type to accurately measure the skin lesion 128. The ruler segmentation module 122 can calculate the number of pixels 127 in the display that measure one millimeter.

The skin lesion area module 124 can be implemented in software running on specialized hardware, full hardware, or a combination thereof. The skin lesion area module 124 accounts for the identification of the skin lesion 128 based on analysis of the pixels 127 in the patient images 114. The skin lesion area rectangle module 124 can analyze each of the skin lesion 128 identified in the patient image 114 to accurately determine the area of the skin lesion 128.

The skin lesion output module 126 can be implemented in software running on specialized hardware, full hardware, or a combination thereof. The skin lesion output module 126 provides an individual skin lesion report that can be utilized by a clinician to plan treatment for the skin lesion 128 identified by the skin lesion area rectangle module 124.

Based on analysis results, the compute system 100 can display information to the patient 112 including a recommendation based on the patient images 114, uploaded, for the patient 112 to schedule a visit with a primary care physician or with a specialist based on the individual skin lesion report.

Continuing the example, the compute system 100 can provide a function that allows the clinician to access the patient images 114 uploaded by the patient 112 and the skin lesion measurement module 116, such as with the web-based dashboard. The compute system 100 allows the clinician to make edits to annotations determined by the skin lesion measurement module 116 and saves the results. The clinician can utilize the skin lesion measurement module 116 to make the diagnostic decision and suggest necessary treatment steps (if applicable).

The compute system 100 can provide guidance to the clinician on the photo guidelines. The image quality checker 118 can accept or reject images for retake based on a pre-specified criteria, such as distance, quality, blur, luminosity, or a combination thereof. Once the patient images 114 are successfully uploaded, the compute system 100 can send or load the patient images 114 to the skin lesion measurement module 116 for analysis.

Continuing the example, the compute system 100 can similarly provide a function that allows the clinician to access the patient images 114 uploaded by the patient 112 and the skin lesion output module 126, such as with the web-based dashboard from the skin lesion measurement module 116. The compute system 100 allows the clinician to make edits to annotations determined by the skin lesion measurement module 116 and saves the results. The clinician can utilize the individual skin lesion report from the skin lesion output module 126 to make the diagnostic decision and takes necessary treatment steps (if applicable).

It has been discovered that the compute system 100 can utilize a U-Net convolutional neural network architecture for the skin lesion measurement module 116 in order to increase accuracy and reproducibility of the skin lesion output module 126. The compute system 100 can calculate the skin lesion area module 124 accurately and repeatably. It is understood that the skin lesion measurement module 116 is shown as part of the second device 106 for simplicity of the description only and the skin lesion measurement module 116 could be implemented in whole or in part in the first device 102.

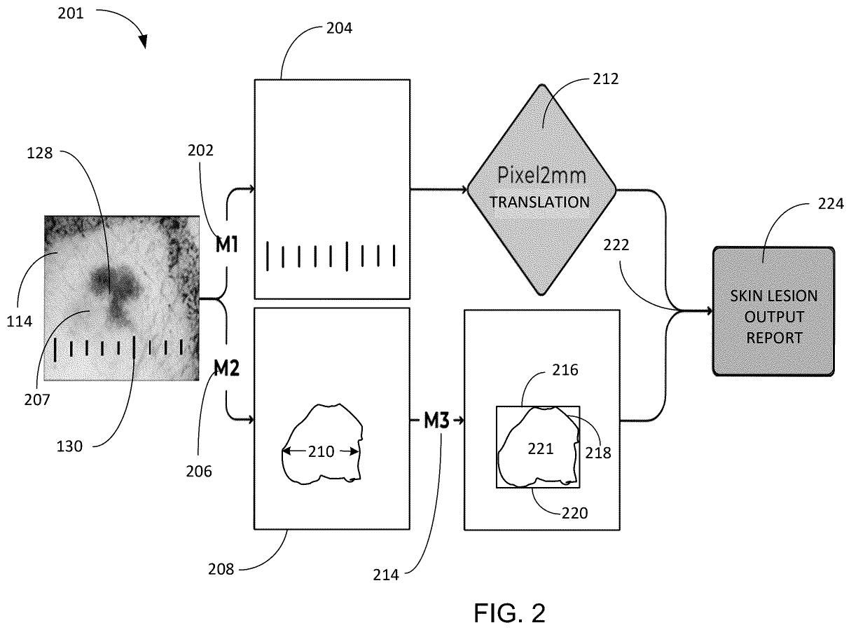

Referring now to FIG. 2, therein is shown an example of a skin lesion area flow 201 including the ruler segmentation module 122, the skin lesion segmentation module 120, and the skin lesion area rectangle module 124. The skin lesion area flow 201 depicts the patient image 114 including the skin lesion 128 and the fixed ruler 130 in preparation for processing. It is understood that the image quality checker 118 would have already allowed the submission of the patient image 114 based on the image quality criteria, such as distance, quality, blur, or a combination thereof with clear visibility of the skin lesion 128, uniform focus throughout, and without visual obstructions, including cosmetics, dirt, or other exogenous pigments.

A ruler segmentation model (M1) 202 can be operated, by the AI of the skin lesion measurement module 116, on the fixed ruler 130. The ruler segmentation model (M1) 202 can correct the dimensions and position of the fixed ruler 130 to generate a correlated ruler 204. Since the fixed ruler 130 is etched on the lens of the dermoscope, it is subject to parallax error and can provide incorrect measurements of the skin lesion 128. The correlated ruler 204 is corrected based on an analysis of the pixel 127 and metadata of the patient image 114. The correlated ruler 204 can position the ruler markings based on the actual size and position of the skin lesion 128 in the patient image 114. It is also understood that the ruler segmentation model (M1) 202 can substitute a different type of the correlated ruler 204 to provide a better measurement of the skin lesion 128.

Concurrently, a skin lesion segmentation model (M2) 206 can be operated, by the AI of the skin lesion measurement module 116, on the skin lesion 128 captured in the patient image 114. The skin lesion segmentation model (M2) 206 can identify the full extent of the skin lesion 128, including a lightly pigmented area 207, to be submitted to a complete lesion image 208. The complete lesion image 208 identifies all of the pigmented area including the lightly pigmented area 207 that makes-up the skin lesion 128. The difference in pigmentation is detected at the level of the pixels 127, which can be missed by the naked eye.

Dermatologist can use a number of approaches to diagnose malignancy of the skin lesion 128. Dermatologists can diagnose malignancy of the skin lesion 128 with “ABCDE rule” for melanoma and the “7 points checklist” (7PCL). The ABCDE acronym stands for asymmetry, border irregularity, color variation, diameter, and evolving. This rule can also emphasize the significance of evolving pigmented lesions in the natural history of melanoma.

On the other hand the 7PCL approach can be used detect features indicating possible melanoma. This approach aims to give a “score” to the skin lesion 128 based on multiple features: change in the size of the skin lesion 128, irregular pigmentation, irregular border, inflammation, itch or altered sensation, a diameter 210 bigger than 7 mm and oozing/crusting of the skin lesion 128. Each feature would score 1 point and skin lesions 128 with scores equal to or bigger than three should be referred for a specialist opinion. The 7PCL can also identify three major signs now scoring 2 points (change in size, shape and/or color) and four minor signs (inflammation, crusting/bleeding, sensory change, the diameter 210 equal or bigger than 7 millimeters).

The correlated ruler 204 can be processed by translating pixel-to-millimeter 212 in order to assure the accurate measurement of the skin lesion 128. The translating pixel-to-millimeter 212 can convert the pixels 127 and metadata of the patient image 114, including the fixed ruler 130, to allow the correlated ruler 204 to indicate the correct value of millimeters in the scale. Thus, the correlated ruler 204 indicates the actual measurement of the skin lesion 128 in millimeters. It is understood that the translating pixel-to-millimeter 212 could be altered to provide other measurement units if desired.

The complete lesion image 208 can be processed by an area estimation model (M3) 214 in order to identify a perimeter outline 218 of the skin lesion 128 and provide a best fit rectangle 216 surrounding the perimeter outline 218. An area calculation module 220 can calculate the area of the best fit rectangle 216 and subtract the space to the perimeter outline 218 in order to calculate a skin lesion area 221 of the skin lesion 128.

The skin lesion output module 126 can overlay skin lesion 128 with the correlated ruler 204 indicating millimeters measurement and the skin lesion area 221 of the skin lesion 128 calculated by the area calculation module 220 to produce a combined output image 222. The skin lesion output module 126 can present a skin lesion output report 224 including the combined output image 222 to the first device 102 of FIG. 1 for display to the patient 112 of FIG. 1 or a Dermatologist (not shown). The skin lesion output report 224 can include the image of the skin lesion 128, the correlated ruler 204, the skin lesion area 221, and suggested actions for the patient 112.

The skin lesion area rectangle module 124 can include the area estimation model (M3) 214 and the area calculation module 220. The ruler segmentation module 122 can include the ruler segmentation model (M1) 202, the correlated ruler 204, and the translating pixel-to-millimeter 212. The skin lesion segmentation module 120 can include skin lesion segmentation model (M2) 206 and the complete lesion image 208.

Embodiments utilize the artificial intelligence (AI) of the skin lesion measurement module 116, as specific examples in the prediction and early detection of cancer through the precise evaluation of the skin lesions 128. Embodiments can analyze vast datasets to identify patterns and anomalies that can indicate the presence of cancer, with greater accuracy and speed than traditional methods. Embodiments provide visual context provided by the correlated ruler 204 displayed on top of a dermoscopic image helps clinicians and AI models interpret features such as size, shape, and growth patterns of the skin lesions 128 with greater accuracy. This contextual understanding provides embodiments for making the skin lesion measurement module 116 decision-making process transparent and comprehensible, aligning with the goals of an explainable artificial intelligence (XAI).

Embodiments continue to advance Deep Learning applied to classification of the skin lesion 128, as neural networks of the skin lesion measurement module 116 can outperform dermatologists. The compute system 100 includes the convolutional neural network (CNN)-based tool of the skin lesion measurement module 116 to assist dermatologists and enhance the detection and treatment of skin diseases. Embodiments of the compute system 100 provide interpretability and the ability to provide an explanation to dermatologists to assist their decision. Moreover, training data themselves contain biases non-meaningful for humans but are exploited by classification models. Understanding and quantifying how much of the decision aligns with medical concepts versus biases indicates the robustness of the compute system 100. An embodiment provides insights into the behavior of the skin lesion measurement module 116 and provide meaningful explanations to practitioners.

Embodiments address visual estimation of the size of the skin lesion 128 that often varies between clinicians and lacks standardized metrics, leading to inconsistent assessments over time. Factors such as lighting conditions, viewing angles, and individual judgment further contribute to potential measurement errors. An evaluation of the dermoscope with the fixed ruler 130 was performed with inputs from four people: a dermatologist, a scientist and two students to estimate the size of the skin lesion 128 on given images, each containing the fixed ruler 130. Skin lesion segmentation can be provided with the image; if they agree with the segmentation, they can estimate the size. Otherwise, they can ignore it. By doing so, embodiments can measure the actual size of the skin lesion 128. Embodiments utilize Intraclass Correlation Coefficient (ICC) to measure the agreement. Embodiments achieved 0.88 ICC score with [0.83, 0.91] of 0.95 confidence interval (CI) among annotators, and an ICC of 0.71 with [0.64, 0.78] of 0.95 confidence interval (CI) between annotators and actual size of moles.

| | Dermatologist | Scientist | Student 1 | Student 2 |
|---|---|---|---|---|
| rMAE/rMSE | 0.24/0.09 | 0.23/0.08 | 0.21/0.07 | 0.25/0.1 |
| ICC (95% CI) | 0.64 (0.5, | 0.55 (0.38, | 0.64 (0.5, | 0.47 (0.29, |
| | 0.75) | 0.68) | 0.75) | 0.62) |

Table 1 depicts relative Mean Absolute Error (rMAE), relative Mean Square Error (rMSE), and ICC of each annotator. Examples of size estimation accuracy of the skin lesions 128 by four people using the fixed ruler 130 on dermoscopic images. The individual ICC score is lower than the group ICC score as in the group, annotators have high correlation. That is, they may make the same error in estimation in some hard cases such as the fixed ruler 130 not aligning with the mole.

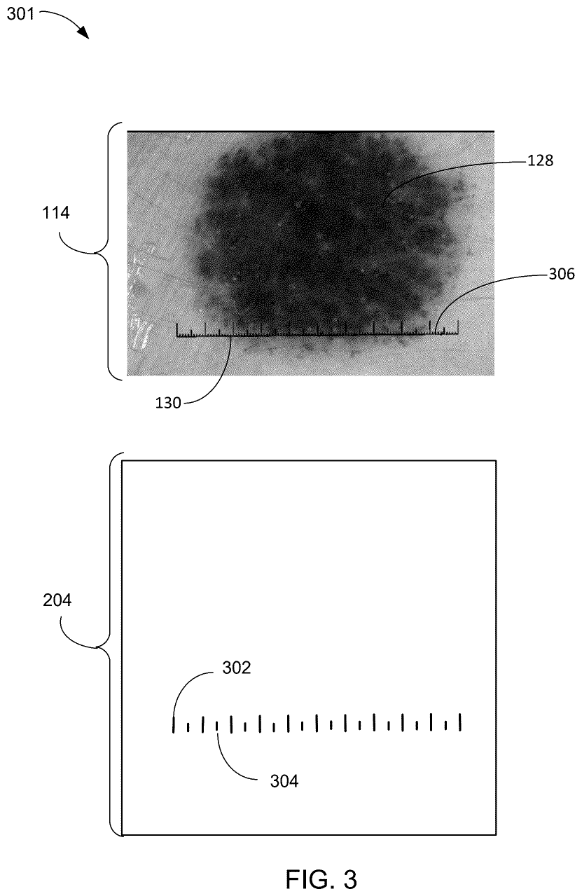

Referring now to FIG. 3, therein is shown an example segmentation 301 of the ruler segmentation module 122 with the patient image 114 input and the correlated ruler 204 as the output. The example segmentation 301 depicts the patient image 114 with the fixed ruler 130 overlapping the skin lesion 128

Regarding the ruler segmentation model (M1) 202 of FIG. 2, as an example, this model can achieve the task of segmenting only major markers 302 and intermediate marks 304 of the fixed rulers 130 present on dermoscopic images. The fixed rulers 130 from different manufacturers exhibit different visual characteristics, including accompanying text, numerical labels, variations in segment lengths, colors, thicknesses, tick marks, etc. The ruler segmentation model (M1) 202 prediction focuses on the location of the major markers 302 and the intermediate marks 304 only, excluding any associated lettering or tick marks 306 of the fixed ruler 130.

The ruler segmentation model (M1) 202 focuses on segmenting the vertical lines 302 of the fixed ruler 130. The ruler segmentation model (M1) 202 can perform an analysis of the pixels 127 and metadata of the patient image 114 in order to detect and reposition the vertical lines 302 appropriately for the scale of the patient image 114.

In this example, the ruler segmentation model (M1) 202 is based on a UNet-like architecture with an EfficientnetB4 encoder pre-trained on ImageNet. Its prediction includes a binary mask using a segmentation head with Sigmoid activation highlighting the fixed ruler 130 in the patient image 114. A Dice loss is used to train the ruler segmentation model (M1) 202. As an example, the Dice loss can provide a measure of the dissimilarity between the predicted segmentation and the true segmentation of an image. An optimizer is used with an initial learning rate of α=5e-3 and a learning rate decay of α×0.998^{ϵ} with ϵ the current epoch. The ruler segmentation model (M1) 202 is trained on images of size 512×512×3 with a batch size of 8. As an example, the optimizer can be short for “Adaptive Moment Estimation,” is an iterative optimization algorithm used to minimize the loss function during the training of neural networks. The optimizer can be looked at as a combination of RMSprop and Stochastic Gradient Descent with momentum.

Embodiments utilize quantitative metrics on the validation set to assess the performance of the ruler segmentation model (M1) 202 during the training and validation phases. The effectiveness of the ruler segmentation model (M1) 202 is evaluated using the Dice loss value and Jaccard similarity coefficient. After 250 epochs of training, embodiments performed with a Dice loss value of 0.0223 and a Jaccard similarity coefficient of 0.9552.

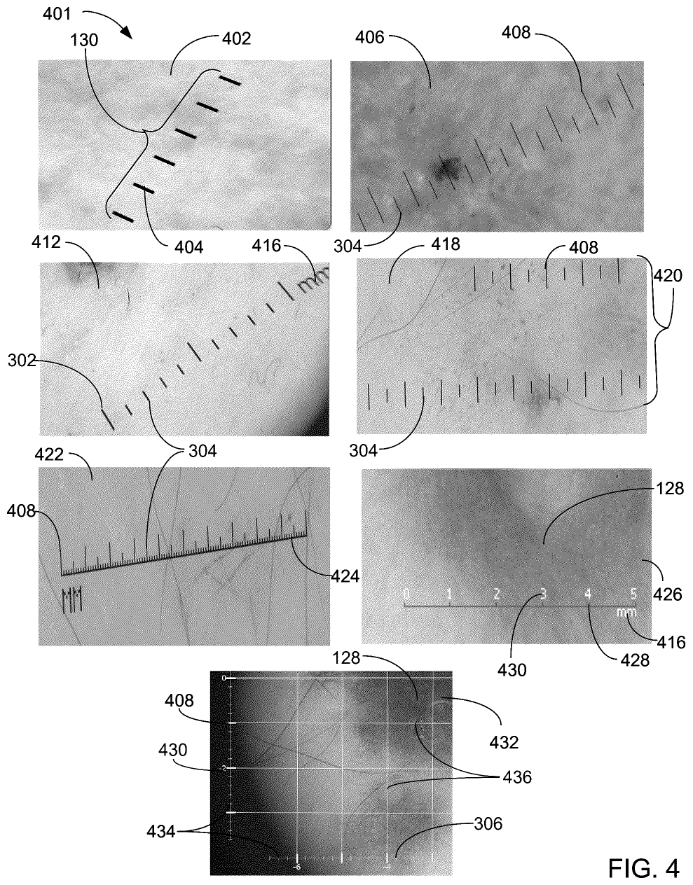

Referring now to FIG. 4, therein is shown examples of fixed ruler types 401 applied to dermoscopic images. The fixed ruler types 401 can provide a suggested reference for measuring the skin lesion 128, but is subject to parallax error causing incorrect measurement.

The fixed ruler types 401 can include a bold scale 402, which depicts a diagonal scale of bold markers 404 without scale indications. The bold scale 402 can provide a relative size indication, but is not suitable for accurate measurement of the skin lesion 128 because of parallax error between the plane of the skin lesion 128 and the plane of the lens that is etched with the fixed ruler 130.

A fine scale 406 can include diagonally positioned fine markers 408 with shorter intermediate markers 304. The fine scale 406 can provide a relative size indication, but is not suitable for accurate measurement of the skin lesion 128 because of parallax error between the plane of the skin lesion 128 and the plane of the lens that is etched with the fixed ruler 130.

An intermediate scale 412 can include diagonally positioned major markers 302 with four of the intermediate markers 304 between them and a scale label 416, such as “mm” for millimeters. The intermediate scale 412 can provide a relative size indication, but is not suitable for accurate measurement of the skin lesion 128 because of parallax error between the plane of the skin lesion 128 and the plane of the lens that is etched with the fixed ruler 130.

A parallel fine scale 418 can include horizontally positioned fine markers 408, in multiple parallel scales 420, with one of the intermediate markers 304 between them. The parallel fine scale 418 can provide a relative size indication, but is not suitable for accurate measurement of the skin lesion 128 because of parallax error between the plane of the skin lesion 128 and the plane of the lens that is etched with the fixed ruler 130.

A detailed fine scale 422 can include diagonally positioning of the fine markers 408 with one of the intermediate markers 304 between them and four tick marks 306 between each of the fine markers 408 and the intermediate markers 304. The scale label 416, such as “MM” for millimeters can also be displayed. The detailed fine scale 422 can provide a relative size indication, but is not suitable for accurate measurement of the skin lesion 128 because of parallax error between the plane of the skin lesion 128 and the plane of the lens that is etched with the fixed ruler 130.

A multicolored scale 426 can include horizontally positioned red tick marks 428 with a numerical marker 430 of a different color, such as white, positioned above the red tick marks 428 and the scale label 416, such as “mm” for millimeters. The multicolored scale 426 can provide a relative size indication, but is not suitable for accurate measurement of the skin lesion 128 because of parallax error between the plane of the skin lesion 128 and the plane of the lens that is etched with the fixed ruler 130.

A grid scale 432 include horizontally and vertically positioned scales 434 each including the fine markers 408 with four of the intermediate markers 304 between them with the numerical marker 430 adjacent to every other of the fine markers 408. Grid lines 436 extend horizontally from the vertical scale 434 and vertically from the horizontal scale 434 forming a grid pattern over the surface of the lens. The grid scale 432 can provide a relative size indication, but is not suitable for accurate measurement of the skin lesion 128 because of parallax error between the plane of the skin lesion 128 and the plane of the lens that is etched with the fixed ruler 130.

It is understood that the application of the scales listed above do not provide sufficient accuracy to analyze the status of the skin lesion 128. It is critical that an accurate measure of the skin lesion 128 be provided in order to monitor growth or shrinking of the skin lesion 128 during prognosis and treatment.

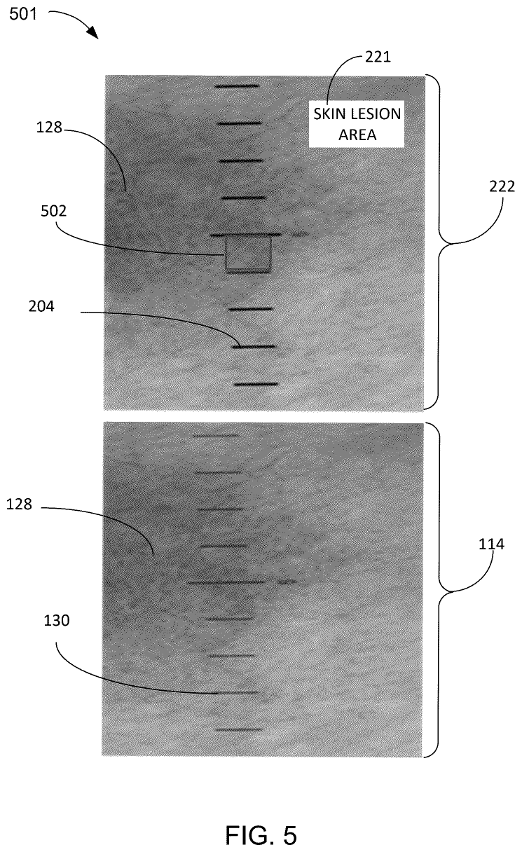

Referring now to FIG. 5, therein is shown an example image 501 for an annotation process that allows training the model of ruler segmentation by the ruler segmentation module 122 of FIG. 1. The example image 501 depicts the patient image 114 including the skin lesion 128 with the fixed ruler 130 overlapping the skin lesion 128.

The ruler segmentation module 122 processes the patient image 114 through the ruler segmentation model (M1) 202 of FIG. 2, in which the AI of the skin lesion measurement module 116 of FIG. 1 can identify the key features of the fixed ruler 130. The ruler segmentation model (M1) 202 can perform the analysis of the pixels 127 and the metadata of the patient image 114 in order to produce the correlated ruler 204, which is correctly positioned on the skin lesion 128, with no parallax error.

The translating pixel-to-millimeter 212 can convert the pixels 127 and the metadata of the patient image 114 to allow the correlated ruler 204 to display a millimeter marker 502 of the measure of one millimeter. The millimeter marker 502 can be displayed or omitted at the patient 112 desires. The combined output image 222 can be provided to the patient 112 by the skin lesion output module 126 of FIG. 1. The combined output image 222 can also indicate a skin lesion area 221 calculated by the skin lesion area rectangle module 124 of FIG. 1, which can include the area estimation model (M3) 214 of FIG. 2 and the area calculation module 220 of FIG. 2.

By way of an example, the processing by the ruler segmentation module 122, for the patient image 114, bounding boxes were designed around the width and height of each line of the fixed ruler 130, which can be converted to binary mask to position the correlated ruler 204. Additionally, the scale from pixels 127 to millimeters was computed for the correlated ruler 204 of each of the patient image 114, ensuring precise measurements necessary for accurate evaluation and algorithm training.

Embodiments can segment the fixed rulers 130 in the patient image 114 and calculate the length of one millimeter in the pixels 127. By incorporating the skin lesion segmentation model (M2) 206 of FIG. 2 and the area calculation module 220 of FIG. 2, which can support a minimum area rectangle algorithm to generate the best fit rectangle 216 of FIG. 2, and determine the skin lesion area 221 or dimensions of the skin lesion 128. An embodiment, as an example, is versatile and can be applied to any type of skin lesion, provided a ruler is present in the image and the lesion segmentation is effective.

In this example, the skin lesion segmentation model (M2) 206 was trained on ISIC images to detect and segment the skin lesions 128 including the lightly pigmented area 207. The implementation of the area calculation module 220 included a UNet architecture was used with encoder EfficientNet B4 with weights pre-trained on ImageNet and input size 320×320×3. An optimizer and binary cross-entropy loss were applied. The models in this example were trained using NVIDIA Titan RTX with a decay learning rate scheduler.

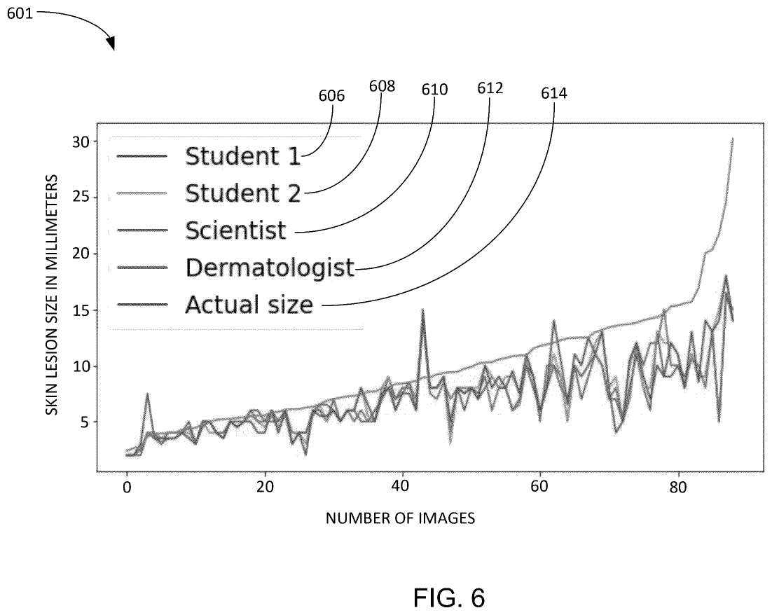

Referring now to FIG. 6, therein is shown an example comparison graph 601 of skin lesion size estimation of 4 different annotators using fixed rulers in a dermoscope. The example comparison graph 601 depicts vertical scale 602, indicating the skin lesion size in millimeters, and a horizontal scale 604 indicating the number of images reviewed by the annotators. The annotators include student 1 606 represented by a blue line, student 2 608 represented by an orange line, a Scientist 610 represented by a green line, and a Dermatologist 612 represented by a red line, while an actual size 614 of the skin lesion 128 of FIG. 1 is represented by a purple line. It is understood that the actual size 614 can be provided by the skin lesion measurement module 116 of FIG. 1 and verified by a group of Dermatologists prior to the annotation of the images by the student 1 606, the student 2 608, the Scientist 610, and the Dermatologist 612.

As can be seen from the comparison graph 601, the first 25 images are able to be tracked fairly well, but once the actual size 614 of the skin lesion 128 reaches about seven millimeters, the annotations become erratic. As the actual size 614 of the skin lesions 128 increases the effects of the parallax error induced by the fixed ruler 130 of FIG. 1 becomes obvious.

Thus, the embodiments of the compute system 100 improves the state of the art in dermatological analysis of the skin lesions 128, which is key to early cancer detection in the case of skin lesions 128, such as melanoma. The actual size 614 of the skin lesion 128 and the correlated ruler 204 provided by the skin lesion measurement module 116 can provide a reliable and accurate indication for Dermatologists and patients 112 of FIG. 1 alike.

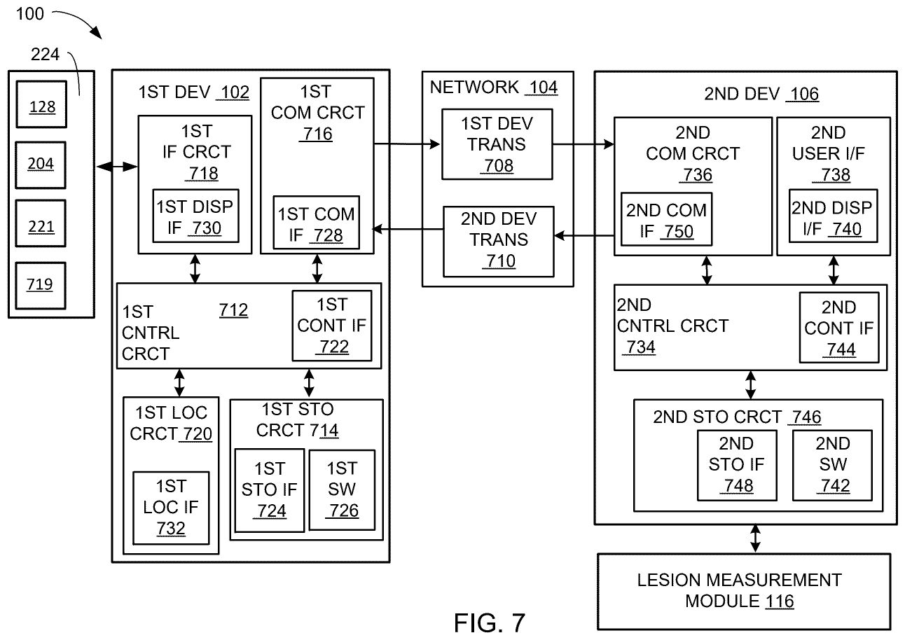

Referring now to FIG. 7, therein is shown an exemplary block diagram of the compute system 100 in an embodiment. The compute system 100 shown in this figure can be utilized to implement the embodiment in one of the devices or distributed or spread across multiple of the devices.

The compute system 100 can include the first device 102, the network 104, and the second device 106. The first device 102 can send information in a first device transmission 708 over the network 104 to the second device 106. The second device 106 can send information in a second device transmission 710 over the network 104 to the first device 102.

For illustrative purposes, the compute system 100 is shown with the first device 102 as a client device, although it is understood that the compute system 100 can include the first device 102 as a different type of device.

Also, for illustrative purposes, the compute system 100 is shown with the second device 106 as a server, although it is understood that the compute system 100 can include the second device 106 as a different type of device. For example, the second device 106 can be a client device. By way of an example, the compute system 100 can be implemented entirely on the first device 102.

Also, for illustrative purposes, the compute system 100 is shown with interaction between the first device 102 and the second device 106. However, it is understood that the first device 102 can be a part of or the entirety of a medical instrument, a smart watch, a smart phone, or a combination thereof. Similarly, the second device 106 can similarly interact with the first device 102 representing the medical device, the smart watch, the smart phone, or a combination thereof.

For brevity of description in this embodiment of the present invention, the first device 102 will be described as a client device and the second device 106 will be described as a server device. The embodiment of the present invention is not limited to this selection for the type of devices. The selection is an example of an embodiment of the present invention.

The first device 102 can include a first control circuit 712, a first storage circuit 714, a first communication circuit 716, a first interface circuit 718, and a first location circuit 720. The first control circuit 712 can include a first control interface 722. The first control circuit 712 can execute a first software 726 to provide the operational intelligence of the compute system 100.

The first control circuit 712 can be implemented in a number of different manners. For example, the first control circuit 712 can be a processor, an application specific integrated circuit (ASIC) an embedded processor, a microprocessor, a hardware control logic, a hardware finite state machine (FSM), a digital signal processor (DSP), or a combination thereof. The first control interface 722 can be used for communication between the first control circuit 712 and other functional units or circuits in the first device 102. The first control interface 722 can also be used for communication that is external to the first device 102.

The first control interface 722 can receive information from the other functional units/circuits or from external sources, or can transmit information to the other functional units/circuits or to external destinations. The external sources and the external destinations refer to sources and destinations external to the first device 102.

The first control interface 722 can be implemented in different ways and can include different implementations depending on which functional units/circuits or external units/circuits are being interfaced with the first control interface 722. For example, the first control interface 722 can be implemented with a pressure sensor, an inertial sensor, a microelectromechanical system (MEMS), optical circuitry, waveguides, wireless circuitry, wireline circuitry, or a combination thereof.

The first storage circuit 714 can store the first software 726. The first storage circuit 714 can also store the relevant information, such as data representing incoming images, data representing previously presented image, sound files, or a combination thereof.

The first storage circuit 714 can be a volatile memory, a nonvolatile memory, an internal memory, an external memory, or a combination thereof. For example, the first storage circuit 714 can be a nonvolatile storage such as non-volatile random-access memory (NVRAM), Flash memory, disk storage, or a volatile storage such as static random-access memory (SRAM).

The first storage circuit 714 can include a first storage interface 724. The first storage interface 724 can be used for communication between the first storage circuit 714 and other functional units or circuits in the first device 102. The first storage interface 724 can also be used for communication that is external to the first device 102.

The first storage interface 724 can receive information from the other functional units/circuits or from external sources, or can transmit information to the other functional units/circuits or to external destinations. The external sources and the external destinations refer to sources and destinations external to the first device 102. The first storage interface 724 can receive input from and source data to the skin lesion measurement module 116. The first storage interface 724 can source the patient image 114 and receive the skin lesion output report 224 in preparation for display to the patient 112 of FIG. 1 on the first device 102.

The first storage interface 724 can include different implementations depending on which functional units/circuits or external units/circuits are being interfaced with the first storage circuit 714. The first storage interface 724 can be implemented with technologies and techniques similar to the implementation of the first control interface 722, including the microelectromechanical system (MEMS), the optical circuitry, the waveguides, the wireless circuitry, the wireline circuitry, or a combination thereof.

The first communication circuit 716 can enable external communication to and from the first device 102. For example, the first communication circuit 716 can permit the first device 102 to communicate with the second device 106 and the network 104.

The first communication circuit 716 can also function as a communication hub allowing the first device 102 to function as part of the network 104 and not limited to be an endpoint or terminal circuit to the network 104. The first communication circuit 716 can include active and passive components, such as microelectronics or an antenna, for interaction with the network 104.

The first communication circuit 716 can include a first communication interface 728. The first communication interface 728 can be used for communication between the first communication circuit 716 and other functional units or circuits in the first device 102. The first communication interface 728 can receive information from the second device 106 for distribution to the other functional units/circuits or can transmit information to the other functional units or circuits.

The first communication interface 728 can include different implementations depending on which functional units or circuits are being interfaced with the first communication circuit 716. The first communication interface 728 can be implemented with technologies and techniques similar to the implementation of the first control interface 722 including the microelectromechanical system (MEMS), the optical circuitry, the waveguides, the wireless circuitry, the wireline circuitry, or a combination thereof.

The first interface circuit 718 allows the patient 112 of FIG. 1 to interface and interact with the first device 102. The first interface circuit 718 can include an input device and an output device. Examples of the input device of the first interface circuit 718 can include a digital camera 719, a keypad, a touchpad, soft-keys, a keyboard, a microphone, an infrared sensor for receiving remote signals, or any combination thereof to provide data and communication inputs. It is understood that the function of he digital camera 719 can be embedded as a portion of the first device 102.

The first interface circuit 718 can include a first display interface 730. The first display interface 730 can include an output device. The first display interface 730 can include a projector, a video screen, a touch screen, a speaker, a microphone, a keyboard, and combinations thereof. The first display interface 730 can present the skin lesion output report 224, including the image of the skin lesion 128, the correlated ruler 204, the skin lesion area 221, and optionally the millimeter marker 502 of FIG. 5.

The first control circuit 712 can operate the first interface circuit 718 to display information generated by the compute system 100 and receive input from the user 112. The first control circuit 712 can also execute the first software 726 for the other functions of the compute system 100, including receiving location information from the first location circuit 720. The first control circuit 712 can further execute the first software 726 for interaction with the network 104 via the first communication circuit 716. The first control circuit 712 can operate the skin lesion measurement module 116 of FIG. 1 in whole or in part.

The first control circuit 712 can also receive location information from the first location circuit 720. The first control circuit 712 can operate the skin lesion measurement module 116.

The first location circuit 720 can be implemented in many ways. For example, the first location circuit 720 can includes hardware that functions as at least a part of the global positioning system, an inertial compute system, a cellular-tower location system, a gyroscope, or any combination thereof. Also, for example, the first location circuit 720 can utilize components such as an accelerometer, gyroscope, or global positioning system (GPS) receiver.

The first location circuit 720 can include a first location interface 732. The first location interface 732 can be used for communication between the first location circuit 720 and other functional units or circuits in the first device 102.

The first location interface 732 can receive information from the other functional units/circuits or from external sources, or can transmit information to the other functional units/circuits or to external destinations. The external sources and the external destinations refer to sources and destinations external to the first device 102. The first location interface 732 can receive the global positioning location from the global positioning system (not shown).

The first location interface 732 can include different implementations depending on which functional units/circuits or external units/circuits are being interfaced with the first location circuit 720. The first location interface 732 can be implemented with technologies and techniques similar to the implementation of the first control circuit 2012.

The second device 106 can be optimized for implementing an embodiment of the present invention in a multiple device embodiment with the first device 102. The second device 106 can provide the additional or higher performance processing power compared to the first device 102. The second device 106 can include a second control circuit 734, a second communication circuit 736, a second user interface 738, and a second storage circuit 746.

The second user interface 738 allows an operator (not shown) to interface and interact with the second device 106. The second user interface 738 can include an input device and an output device. Examples of the input device of the second user interface 738 can include a keypad, a touchpad, soft-keys, a keyboard, a microphone, or any combination thereof to provide data and communication inputs. Examples of the output device of the second user interface 738 can include a second display interface 740. The second display interface 740 can include a display, a projector, a video screen, a speaker, or any combination thereof.

The second control circuit 734 can execute a second software 742 to provide the intelligence of the second device 106 of the compute system 100. The second software 742 can operate in conjunction with the first software 726. The second control circuit 734 can provide additional performance compared to the first control circuit 712.

The second control circuit 734 can operate the second user interface 738 to display information. The second control circuit 734 can also execute the second software 742 for the other functions of the compute system 100, including operating the second communication circuit 736 to communicate with the first device 102 over the network 104.

The second control circuit 734 can be implemented in a number of different manners. For example, the second control circuit 734 can be a processor, an embedded processor, a microprocessor, hardware control logic, a hardware finite state machine (FSM), a digital signal processor (DSP), or a combination thereof.

The second control circuit 734 can include a second control interface 744. The second control interface 744 can be used for communication between the second control circuit 734 and other functional units or circuits in the second device 106. The second control interface 744 can also be used for communication that is external to the second device 106.

The second control interface 744 can receive information from the other functional units/circuits or from external sources, or can transmit information to the other functional units/circuits or to external destinations. The external sources and the external destinations refer to sources and destinations external to the second device 106.

The second control interface 744 can be implemented in different ways and can include different implementations depending on which functional units/circuits or external units/circuits are being interfaced with the second control interface 744. For example, the second control interface 744 can be implemented with a pressure sensor, an inertial sensor, a microelectromechanical system (MEMS), optical circuitry, waveguides, wireless circuitry, wireline circuitry, or a combination thereof.

The second storage circuit 746 can store the second software 742. The second storage circuit 746 can also store the information such as data representing incoming images, data representing previously presented image, sound files, or a combination thereof. The second storage circuit 746 can be sized to provide the additional storage capacity to supplement the first storage circuit 714.

For illustrative purposes, the second storage circuit 746 is shown as a single element, although it is understood that the second storage circuit 746 can be a distribution of storage elements. Also, for illustrative purposes, the compute system 100 is shown with the second storage circuit 746 as a single hierarchy storage system, although it is understood that the compute system 100 can include the second storage circuit 746 in a different configuration. For example, the second storage circuit 746 can be formed with different storage technologies forming a memory hierarchal system including different levels of caching, main memory, rotating media, or off-line storage.

The second storage circuit 746 can be a controller of a volatile memory, a nonvolatile memory, an internal memory, an external memory, or a combination thereof. For example, the second storage circuit 746 can be a controller of a nonvolatile storage such as non-volatile random-access memory (NVRAM), Flash memory, disk storage, or a volatile storage such as static random access memory (SRAM).

The second storage interface 748 can receive information from the other functional units/circuits or from external sources, or can transmit information to the other functional units/circuits or to external destinations. The external sources and the external destinations refer to sources and destinations external to the second device 106.

The second storage interface 748 can include different implementations depending on which functional units/circuits or external units/circuits are being interfaced with the second storage circuit 746. The second storage interface 748 can be implemented with technologies and techniques similar to the implementation of the second control interface 744.

The second communication circuit 736 can enable external communication to and from the second device 106. For example, the second communication circuit 736 can permit the second device 106 to communicate with the first device 102 over the network 104.

The second communication circuit 736 can also function as a communication hub allowing the second device 106 to function as part of the network 104 and not limited to be an endpoint or terminal unit or circuit to the network 104. The second communication circuit 736 can include active and passive components, such as microelectronics or an antenna, for interaction with the network 104.

The second communication circuit 736 can include a second communication interface 750. The second communication interface 750 can be used for communication between the second communication circuit 736 and other functional units or circuits in the second device 106. The second communication interface 750 can receive information from the other functional units/circuits or can transmit information to the other functional units or circuits.

The second communication interface 750 can include different implementations depending on which functional units or circuits are being interfaced with the second communication circuit 736. The second communication interface 750 can be implemented with technologies and techniques similar to the implementation of the second control interface 744.

The second communication circuit 736 can couple with the network 104 to send information to the first device 102. The first device 102 can receive information in the first communication circuit 716 from the second device transmission 710 of the network 104. The compute system 100 can be executed by the first control circuit 712, the second control circuit 734, or a combination thereof.

For illustrative purposes, the second device 106 is shown with the partition containing the second user interface 738, the second storage circuit 746, the second control circuit 734, and the second communication circuit 736, although it is understood that the second device 106 can include a different partition. The second control circuit 734 can operate the skin lesion measurement module 116 in whole or in part to support the compute system 100. For example, the second software 742 can be partitioned differently such that some or all of its function can be in the second control circuit 734 and the second communication circuit 736. Also, the second device 106 can include other functional units or circuits not shown in FIG. 1 for clarity.

The functional units or circuits in the first device 102 can work individually and independently of the other functional units or circuits. The first device 102 can work individually and independently from the second device 106 and the network 104.

The functional units or circuits in the second device 106 can work individually and independently of the other functional units or circuits. The second device 106 can work individually and independently from the first device 102 and the network 104.

The functional units or circuits described above can be implemented in hardware. For example, one or more of the functional units or circuits can be implemented using a gate array, an application specific integrated circuit (ASIC), circuitry, a processor, a computer, integrated circuit, integrated circuit cores, a pressure sensor, an inertial sensor, a microelectromechanical system (MEMS), a passive device, a physical non-transitory memory medium containing instructions for performing the software function, a portion therein, or a combination thereof.

For illustrative purposes, the compute system 100 is described by operation of the first device 102 and the second device 106. It is understood that the first device 102 and the second device 106 can operate any of the modules and functions of the compute system 100.

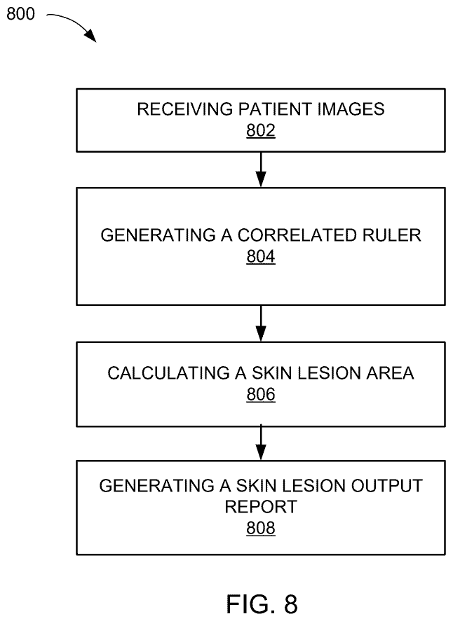

Referring now to FIG. 8, therein is shown is a flow chart of a method of operation 800 of a compute system 100 in an embodiment of the present invention. The method 800 includes: receiving a patient image including a skin lesion and a fixed ruler in a block 802; generating a correlated ruler by segmenting the fixed ruler including translating pixels-to-millimeter of the patient image in a block 804; calculating a skin lesion area by segmenting the skin lesion on a block 806; and generating a skin lesion output report, including an image of the skin lesion, the correlated ruler and the skin lesion area, for displaying on a device in a block 808.

The resulting method, process, apparatus, device, product, and/or system is straightforward, cost-effective, uncomplicated, highly versatile, accurate, sensitive, and effective, and can be implemented by adapting known components for ready, efficient, and economical manufacturing, application, and utilization. Another important aspect of an embodiment of the present invention is that it valuably supports and services the historical trend of reducing costs, simplifying systems, and increasing performance.

These and other valuable aspects of an embodiment of the present invention consequently further the state of the technology to at least the next level.

While the invention has been described in conjunction with a specific best mode, it is to be understood that many alternatives, modifications, and variations will be apparent to those skilled in the art in light of the foregoing description. Accordingly, it is intended to embrace all such alternatives, modifications, and variations that fall within the scope of the included claims. All matters set forth herein or shown in the accompanying drawings are to be interpreted in an illustrative and non-limiting sense.

What is claimed is:

1. A method of operation of a compute system comprising:
receiving a patient image including a skin lesion and a fixed ruler;
generating a correlated ruler by segmenting the fixed ruler including translating pixels-to-millimeter of the patient image;
calculating a skin lesion area by segmenting the skin lesion; and
generating a skin lesion output report, including an image of the skin lesion, the correlated ruler and the skin lesion area, for displaying on a device.

2. The method as claimed in claim 1 further comprising displaying a millimeter marker on the correlated ruler of the skin lesion output report.

3. The method as claimed in claim 1 wherein generating the correlated ruler includes operating a ruler segmentation model (M1), previously trained, on the fixed ruler to detect only a major marker and an intermediate mark.

4. The method as claimed in claim 1 wherein calculating the skin lesion area includes identifying a perimeter outline of the skin lesion.

5. The method as claimed in claim 1 wherein calculating the skin lesion area includes identifying a best fit rectangle surrounding a perimeter outline of the skin lesion.

6. The method as claimed in claim 1 further comprising generating a lesion image of the skin lesion area by analyzing of the pixels to identify a lightly pigmented area as part of the skin lesion.

7. The method as claimed in claim 1 further comprising identifying an actual size of the skin lesion with the correlated ruler and the skin lesion area analyzed to the level of the pixels.

8. A compute system comprising:
a control circuit, including a processor, configured to:
capture a patient image, through a digital camera, including a skin lesion and a fixed ruler;
generate a correlated ruler by segmenting the fixed ruler including translating pixels-to-millimeter of the patient image;
calculate a skin lesion area by segmenting the skin lesion; and
generate a skin lesion output report, including an image of the skin lesion, the correlated ruler and the skin lesion area, for displaying on a device.

9. The system as claimed in claim 8 wherein the control circuit further configured to display a millimeter marker, provided by the translating pixel-to-millimeter on the correlated ruler of the skin lesion output report.

10. The system as claimed in claim 8 wherein the control circuit configured to generate the correlated ruler includes a ruler segmentation model (M1), previously trained, operated on the fixed ruler to detect only a major marker and an intermediate mark.

11. The system as claimed in claim 8 wherein the control circuit configured to calculate the skin lesion area includes an area estimation model (M3), previously trained, applied to identify a perimeter outline of the skin lesion.

12. The system as claimed in claim 8 wherein the control circuit configured to calculate the skin lesion area includes an area estimation model (M3), previously trained, applied to identify a best fit rectangle surrounding a perimeter outline of the skin lesion.

13. The system as claimed in claim 8 wherein the control circuit configured to generate a lesion image of the skin lesion area by a skin lesion segmentation model (M2), previously trained, applied to perform an analysis of the pixels to identify a lightly pigmented area as part of the skin lesion.

14. The system as claimed in claim 8 wherein the control circuit configured to identify an actual size of the skin lesion by the correlated ruler and the skin lesion area analyzed to the level of the pixels.

15. A non-transitory computer readable medium including instructions executable by a control circuit for a compute system performing functions comprising:
capturing a patient image including a skin lesion and a fixed ruler;
generating a correlated ruler by segmenting the fixed ruler including translating pixel-to-millimeter of the patient image;
calculating a skin lesion area by segmenting the skin lesion; and
generating a skin lesion output report, including an image of the skin lesion, the correlated ruler and the skin lesion area, for displaying on a device.

16. The non-transitory computer readable medium as claimed in claim 15 further comprising displaying a millimeter marker, provided by the translating pixel-to-millimeter on the correlated ruler of the skin lesion output report.

17. The non-transitory computer readable medium as claimed in claim 15 wherein generating the correlated ruler includes operating a ruler segmentation model (M1), previously trained, on the fixed ruler to detect only a major marker and an intermediate mark.

18. The non-transitory computer readable medium as claimed in claim 15 wherein calculating the skin lesion area includes applying an area estimation model (M3), previously trained, identify a perimeter outline of the skin lesion.

19. The non-transitory computer readable medium as claimed in claim 15 further comprising generating a lesion image of the skin lesion area by applying a skin lesion segmentation model (M2), previously trained, to perform an analysis of the pixels to identify a lightly pigmented area as part of the skin lesion.

20. The non-transitory computer readable medium as claimed in claim 15 further comprising identifying an actual size of the skin lesion by the correlated ruler and the skin lesion area analyzed to the level of the pixels.
